(19) United States
(12) Patent Application Publication
(10) Pub. No.: US 2021/0308314 A1
(43) Pub. Date: Oct. 7, 2021

(54) Fragrance Carrier

(71) Applicant: Nima Sedighi, Plano, TX (US)

(72) Inventor: Nima Sedighi, Plano, TX (US)

(21) Appl. No.: 16/842,398

(22) Filed: Apr. 7, 2020

(51) Int. Cl. A61L 9/12

(52) U.S. Cl. CPC A61L 9/12; A61L 2209/14

(57) ABSTRACT

A mechanism for holding a scented liquid capable of being carried on a person and discretely placed on the user, such as in a pocket, clipped to a person or worn on a chain. The mechanism, when activated by the user, allows for a flow of scent from the liquid to be either automatically heated or organically released in small quantities over time thereby allowing the user to keep a consistent scent on his or her person. The scent is released when heated by a heating means, or through organic contact with body heat, and slowly allowed to evaporate thereby dispersing the scent selected by the user.

### CROSS-REFERENCE TO RELATED APPLICATIONS

None.

### TECHNICAL FIELD

The present apparatus relates generally to aroma and other scented devices meant to be carried on a person.

### RELEVANT PATENT PRIOR ART REFERENCES

### Background

For centuries, fragrances, perfumes, and scented materials have been widely used by both men and women primarily by directly applying such materials to the human body or clothing. Other devices focus on scented objects for automobile, shoes, purses, bags, but they do not address or create a consistently released and operable scent connected to the body itself. The present invention is uniquely different from other perfumed articles, air fresheners, and sachets in that it is worn on the body and the aroma can be adjusted depending on the needs of the person. The present invention offers the pleasure and therapeutic benefits of scented materials worn on the body in lieu of directly spraying clothing or the body with a fragrance. Importantly, this invention has a unique medical purpose as people are often allergic to scented materials, oils or perfumes that come into direct contact the body either on the skin or through clothing. The present invention allows the user the benefit of the enjoying a scent with none of the risks of a contact allergy.

### SUMMARY

In accordance with the invention, a container 6 for holding a scented liquid 4 capable of being carried on a person in a discrete location, such as in a pocket or worn on a chain. The scented liquid 4 contained with container 6 can be aromatically heated by a heater or the warmth of a body and the scent can escape the container through a ventilation grate 1 at the top of the container. The container 6 can also be closed using a closing mechanism such that the grates are closed, and the scent no longer allowed to escape the container. Beneath the container grate 1 and container grate 16 exist a dispersing filter that allows scent to escape when evaporated but prevents the scented liquid 5 and scented liquid 15 from escaping the container. The dispersing filter can be made from paper, cellulose products, plastic, foam or equivalent materials and extend the width of the grate.

In an alternative version, the scented liquid can be replaced with a solid substrate infused with a non-aqueous scented liquid. A scented liquid can be oil or other fragrance, or infused oil or other fragrance within the non-aqueous scented liquid. The solid substrate can be paper, cellulose products, foam or equivalent materials.

The present invention is designed to be worn near the body, but the scented materials do not come into direct contact with the body or skin.

### BRIEF DESCRIPTION OF THE DRAWINGS

FIG. 1 is a side view of an embodiment of the scent device wherein the grate holes are open by opening mechanism 2 slidably adjusting across grate adjuster 3 to allow the scent 4 to be diffused through open grate 1.

FIG. 2 is top view of moveable grate of the scent device in the open position, which opens and closes based on a user adjusting the opening mechanism 2, allowing grate holes 8 to open by opening mechanism 2 slidably adjusting within mechanism 7.

FIG. 3 is top view of a moveable grate of the scent device in the closed position, which opens and closes based on a user adjusting the opening mechanism 2, with grate holes 9 closed by opening mechanism 2.

FIG. 4 is side view of an embodiment of the scent device showing the opening mechanism 12 by rotating cover 10 about the top axis 11 causing the scent fluid scent to disperse through the grate holes 16. As more fully show in FIG. 5, the battery 18 inserted through the removable floor 19 with optional screw cover 17 for the purposed of engaging a heating element.

FIG. 5 is a cross section view of an embodiment of the scent device showing a heating element powered by a removeable batter causing the scent fluid 23 to be heated for evaporation and aromatic dispersion through the grate holes 25 if top 24 has been rotated to the open position. The heating element 21 is powered by the battery connectors 20 when a battery 18 is inserted through the removable battery cover 19.

FIG. 6 is top view of a moveable grate of the scent device in the open position, which opens and closes based on a user adjusting the opening mechanism 12 by rotating cover 10 about the top axis 11 causing the scent fluid scent to disperse through the grate holes 16.

FIG. 7 is top view of a moveable grate of the scent device in the closed position, which opens and closes based on a user adjusting the opening mechanism 12 closing by securing mechanism 13 by rotating cover 10 about the top axis 11 causing the scent to be contained by cover 10.

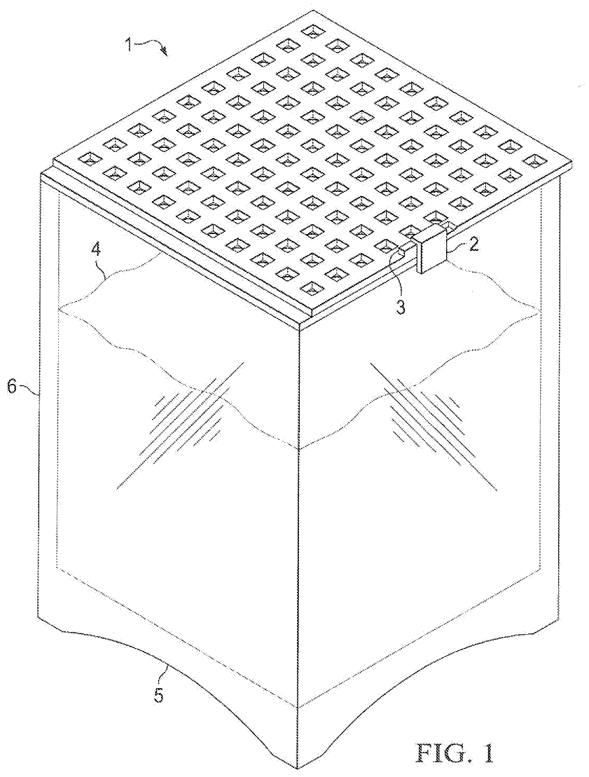

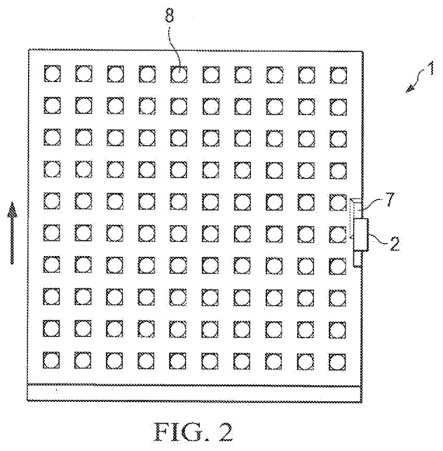

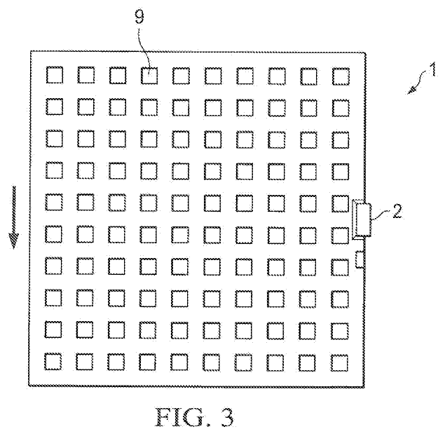

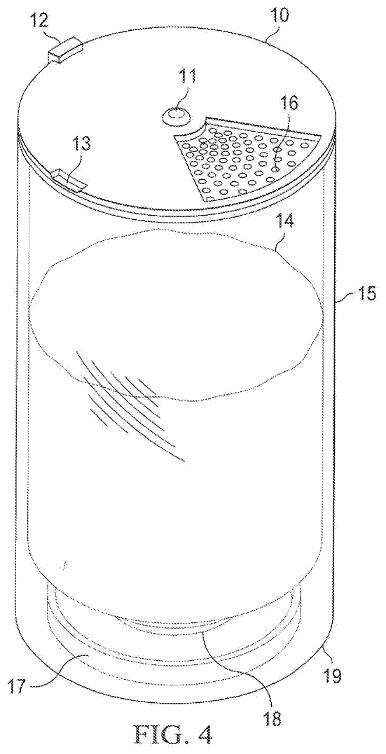

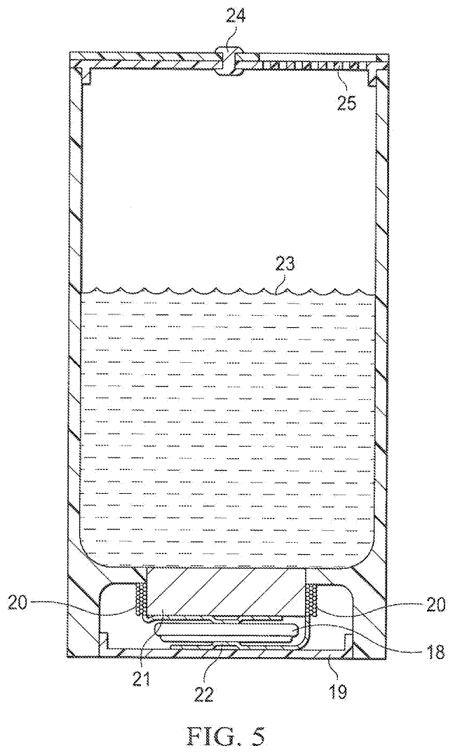

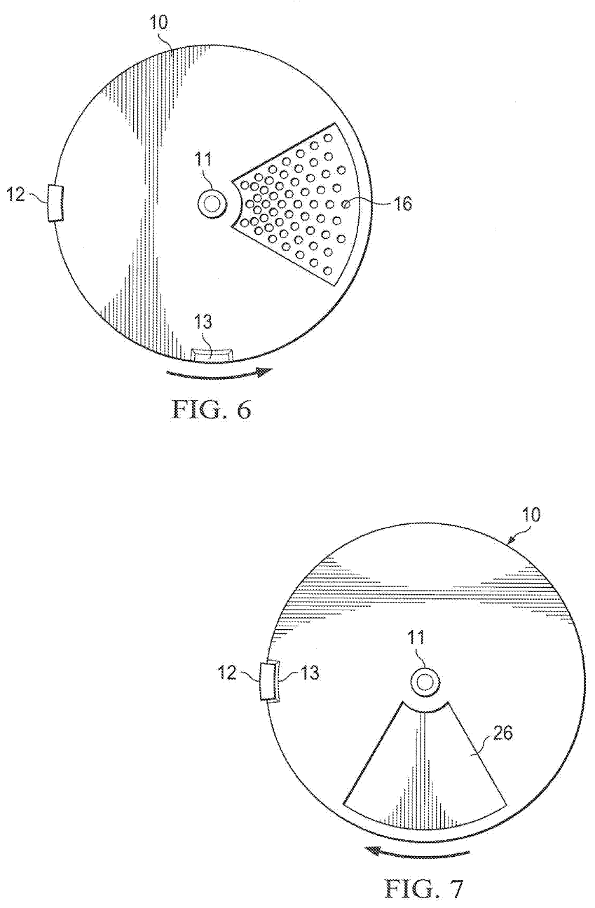

### SPECIFICATION

The present invention consists of a container 6 for storing a scented material 4. The container has a top and bottom, and an adjustable top cover 1. Within the adjustable top cover, there are many openings 8 which open and close based on the position of the adjustable top cover. The plethora of openings allow scent to escape if open or lessen if closed. Each top cover can be opened using some basic means such as a slidably hinged cap, lock, latch, clip, or hinged magnets located on the underside of the adjustable top cover. The hinges, cap, lock, latch and clip can be affixed anywhere the edge of the top cover such that the opening is exposed and closed.

In the event the scented material is a liquid, gel, perfume, oil or other water-based material, then a dispersing filter will exist beneath the top cover to prevent leakage but still allow scent to escape the container. The dispersing filter acts a barrier to prevent the fluid from leaking from the top of the container, and would consist of materials such as foam, plastic, paper, cellulose or other like materials capable of retaining the fluid but allowing the scent to escape the container.

Alternatively, the scented materials can be an infused solid substrate rather than just a liquid. In that case, the dispersing filter is optional as there is no concern over leakage from the container. The infused scented material could consist of a cloth, foam or cellulose materials soaked in an oil, perfume, or scented aqueous material.

In the above embodiments as to both scented and infused scented materials, the present invention could use body heat or natural ambient temperature of the environment to allow the scented material or infused scented material to create an aroma that dissipates from an open container. In an alternative version, the aromatic personal freshener may include a removeable container bottom or bottom cap, a battery storage compartment disposed within the container bottom, a battery, and a heater element which when powered heats the scented and infused scented materials thereby strengthening the scent dispersion. The circuit for turning on the heating element would be operatively connected to the container's adjustable cover top thereby turning on when the container's cover top is opened exposing the ventilation grate to the environment.

In an additional embodiment, the aromatic personal freshener can be either work in a pocket such as pants pocket or shirt pocket or be otherwise fastened to a person via a carabiner, clip, chain, or pin or other like device.

The foregoing merely illustrates the principles of the present invention. Therefore, it will be appreciated that those skilled in the art will be able to devise numerous alternative arrangements that, while not shown or described herein, embody the principles of the invention and thus are within the spirit and scope of the invention.

I claim:

1. An aromatic personal freshener, comprising:
a container for storing a scented material;
said container having a top and a bottom;
an adjustable top cover of said container top;
a plethora of openings dispersed across said adjustable top cover;
a mechanism for opening and closing said adjustable top cover wherein said openings can be covered and uncovered.

2. The aromatic personal freshener of claim 1 wherein a dispersing filter is fitted between said adjustable top cover and said container top.

3. The aromatic personal freshener of claim 2 wherein the scented material is selected from the group consisting of gel, oil, perfume, and scented aqueous materials.

4. The aromatic personal freshener of claim 1 or claim 2 wherein the scented material is an infused solid substrate.

5. The aromatic personal freshener of claim 4 wherein the infused scented material is selected from the group consisting of gel, oil, perfume, and scented aqueous materials.

6. The aromatic personal freshener of claim 3 or 5 wherein the mechanism for opening and closing said adjustable top is selected from the group consisting of a slidably hinged cap, lock, latch, clip, and magnets.

7. The aromatic personal freshener of claim 1 or 6 wherein said aromatic personal freshener container bottom includes a removable cap, a battery storage compartment, a battery, and a heater element operably connected to said battery for the purpose of heating said scented material when the adjustable to cover it opened.

8. The aromatic personal freshener of claim 2 wherein the dispersing filter is selected from the group consisting of foam, plastic, paper and cellulose.

9. The aromatic personal freshener of claim 7 wherein the aromatic personal freshener includes a fastening device for securing said personal freshener to a person's clothing.

10. The aromatic personal freshener of claim 9 wherein said fastening device is selected from the group consisting of clip, chain, pin, Velcro, and carabiner.
